(19) United States
(12) Patent Application Publication
(10) Pub. No.: US 2006/0259025 A1
(43) Pub. Date: Nov. 16, 2006

(54) Conductive fluid bridge electrosurgical apparatus

(75) Inventor: Robert H. Dahla, Sunnyvale, CA (US)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/130,931

(22) Filed: May 16, 2005

(51) Int. Cl. A61B 18/14

(52) U.S. Cl. 606/41

(57) ABSTRACT

An electrosurgical apparatus for treating body tissue, comprising an active and a return electrode, a vacuum inlet located near the active electrode, and at least one pinhole defined on the apparatus near the return electrode that is adapted to provide a conductive fluid bridge between the active and return electrodes during use regardless of the orientation of the electrodes relative to the tissue, without flooding the electrodes or the tissue. Also, a method and system of performing a dry field surgical procedure comprising applying ablative energy to a target tissue wherein the target tissue is not flooded or submerged in electrically conductive fluid; and maintaining a fluid bridge between the electrodes regardless of the orientation of the shaft. Advantageously, since the conductive fluid bridge is maintained for any orientation of the electrodes relative to the tissue without flooding, the instrument can be used to treat tissue from any orientation without breaking the fluid bridge.

### BACKGROUND

1. Field of the Invention

This invention relates to an electrosurgical apparatus comprising an active electrode, a return electrode, and a conductive fluid bridge maintained between the electrodes during use regardless of the orientation of the apparatus.

2. Description of the Prior Art

In some electrosurgical procedures an instrument, as illustrated for example in FIG. 1, comprising an active electrode (12) and a return electrode (14) is used to treat body tissue. Treatment includes without limitation coagulation, cutting, ablating, abrading or puncturing the tissue. In various embodiments, generating plasma between the electrodes and applying the plasma to the tissue, without passing a current through the tissue, effects treatment. Examples of these instruments and their use in electrosurgical procedures are described in U.S. Pat. Nos. 5,683,366 and 6,235,020 herein incorporated by reference. In various embodiments, the conductive fluid path is an electrolyte e.g., saline, lactated ringers solution, or conductive gels, and one electrode, referred to as the active electrode, is designed to generate a higher current density relative to other electrode, referred to as the return electrode. The source of the current is a high frequency voltage applied across the electrodes.

With these instruments, for certain procedures it is a problem to maintain an unbroken conductive fluid path between the electrodes during use. For example as shown in FIG. 2(a), when the instrument is being used to treat tissues (16) in the nose, gravity tends to cause the fluid bridge to break. Similarly, as shown in FIG. 2(b) the fluid bridge is easily broken when the electrodes (18) are oriented downwards during use. In these procedures because the distal end of the instrument is positioned either higher than the proximal end and/or the electrodes are turned up during use, gravity causes the fluid to flow downwards and away from the electrodes, thus breaking the fluid path. With the breaking of the fluid path, the instrument may exhibit undesirable thermal generation as opposed to the desired generation of plasma.

An approach to maintaining the conductive fluid path during use regardless of the orientation of the instrument is to direct a flow of conductive fluid between the electrodes such that the electrodes and or the tissues are always flooded. This is illustrated for example in FIGS. 3 (a) and (b) wherein a stream of fluid (20) bathes or floods the electrodes (22, 24) during use. In various embodiments, the fluid is discharged from an annular member onto the tissue and/or between the electrodes. In various embodiments, the annular member is an open-ended tube (17) disposed within the instrument as is shown for example in FIG. 1; in other embodiments, the annular member resides externally on an elongate member (11) of the instrument. In various embodiments a vacuum is positioned near the fluid path to aspirate excess fluid during use.

While the fluid path from an annular member is relative easy to maintain by flooding the tissue and/or by ensuring that the tissue is below the level of the electrodes, in using the instruments where flooding is not possible and/or desired as, for example in treating the larynx and the nose, this approach is unsatisfactory. Further, the flooding may be undesirable if it obstructs the line of sight to the tissue during use.

Accordingly, in view of these deficiencies and also in view of the desire to improve the instrument, it is an objective of this invention to provide an instrument wherein the conductive fluid path is maintained during use regardless of the orientation of the electrodes relative to the tissue.

### SUMMARY OF THE INVENTION

In one embodiment, the apparatus comprises an electrosurgical instrument for treating body tissue, comprising an active and a return electrode, a vacuum suction inlet located near the active electrode, and

a pinhole near the return electrode such that the pinhole and the vacuum suction cooperate to maintain a conductive fluid bridge between the active and return electrodes during use, regardless of the orientation of the instrument relative to the tissue.

In another embodiment the apparatus is an electrosurgical instrument comprising an elongated shaft having a distal end portion, an active and a return electrode disposed on the distal end portion, a vacuum system having a suction inlet near the active electrode, and at least one pinhole near the return electrode for forming an conductive fluid bridge between the active and return electrodes during use regardless of the orientation of the instrument.

In another embodiment the invention is a method of ablating body tissue, comprising applying ablative energy to a target tissue not flooded or submerged with an electrically conductive fluid; maintaining an electrically conductive fluid bridge between an active and a return electrode near the target tissue to generate the ablative energy regardless of the orientation of the electrodes relative to the target tissue. In one embodiment the conductive fluid bridge is maintained during use by causing the fluid to flow out of a pinhole placed on the distal end of a shaft towards an aspiration port on the shaft.

In another embodiment the invention is a system for ablating tissue comprising an apparatus including a pinhole for maintaining an electrically conductive fluid bridge between an active and a return electrode on the apparatus regardless of the orientation of the electrodes relative to the tissue; a vacuum system for aspirating fluid from the fluid bridge; a high-frequency voltage generator for generating plasma between the active and return electrodes; and a conductive fluid reservoir system for maintaining a supply of conductive fluid at the electrodes.

In the various embodiments, the instrument maintains a conductive fluid bridge between the electrodes during use regardless of the orientation of the electrodes relative to the target tissue. The fluid bridge comprises an electrically conductive fluid in the form of a droplet, a fluid channel, a glob of gel between the electrodes, etc. An example of a fluid bridge in accordance with the present invention is illustrated in FIG. 5 wherein a bridge (42) is illustrated between the active electrode (34) and a return electrode (36). By directing the conductive fluid through a pinhole near the electrodes, and by operating vacuum aspiration port near the electrodes in accordance with the invention, a conductive fluid bridge of a proper size is maintained during use regardless of the orientation of the instrument relative to the tissue.

Advantageously, since the pinhole of the present instrument restricts the amount of fluid forming the fluid bridge during use, the size of the fluid bridge is restricted. Thus with the vacuum suction inlet located on the instrument, in use the fluid bridge is maintained regardless of the orientation of the instrument with respect to the tissue, without flooding the electrodes and/or the tissue. Accordingly the present instrument is usable to treat tissue for any orientation of the electrodes relative to the tissue, without loss of plasma generation. Additionally the instrument can be used in procedures wherein it is neither desirable nor possible to flood the tissue. Further, since the pinhole restricts the amount of conductive fluid between the electrodes compared to the amount of fluid from an annular orifice on conventional instruments, the instrument allows for better visibility during use.

Without desiring to be bound to any explanation for the results achieved, it is believed that in accordance with the present instrument since fluid emerging from the pinhole creates a small fluid bridge between the electrodes, the surface tension forces arising from the geometry of the instrument and the fluid, in combination with negative pressure of the vacuum and the fluid momentum, counteract the effect of gravity such that the fluid bridge is maintained between the electrodes during use, regardless of the orientation of the instrument.

### BRIEF DESCRIPTION OF THE DRAWINGS

FIG. 1 is a perspective view of an embodiment of an electrosurgical apparatus.

FIGS. 2a and 2b are cross-sectional views of target tissue treatable with an electrosurgical apparatus.

FIGS. 3a and 3b are cross-section views of prior art instruments illustrating a supply of conductive fluid to the electrodes.

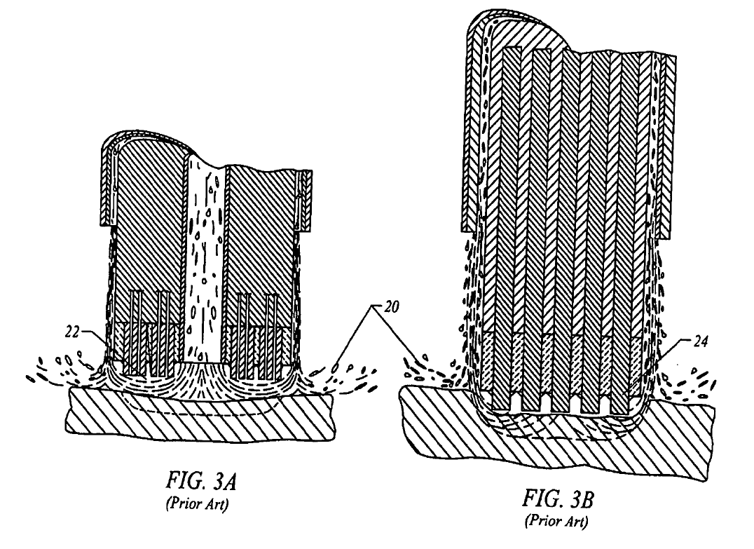

FIG. 4 is a perspective view of an environment for using the present apparatus.

FIG. 5 is a cross-sectional view of an embodiment of a pinhole for forming a fluid bridge between the electrodes without flooding.

### DETAILED DESCRIPTION OF A PREFERRED EMBODIMENT

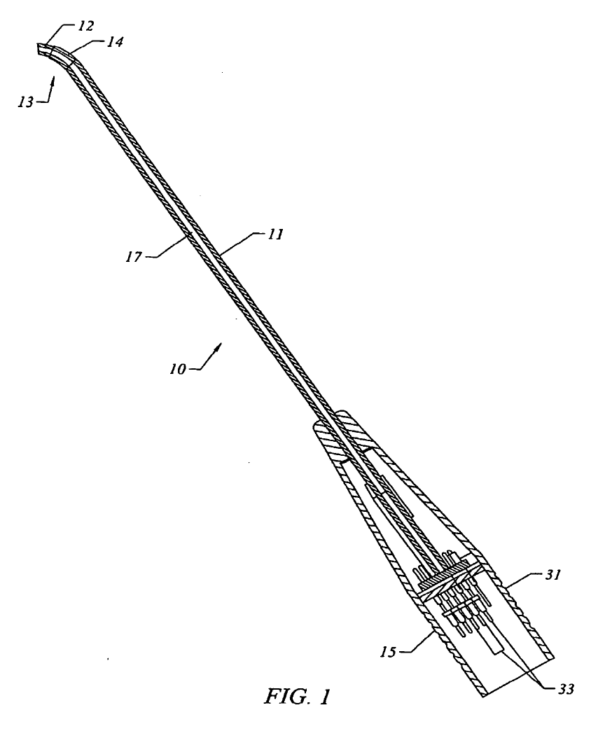

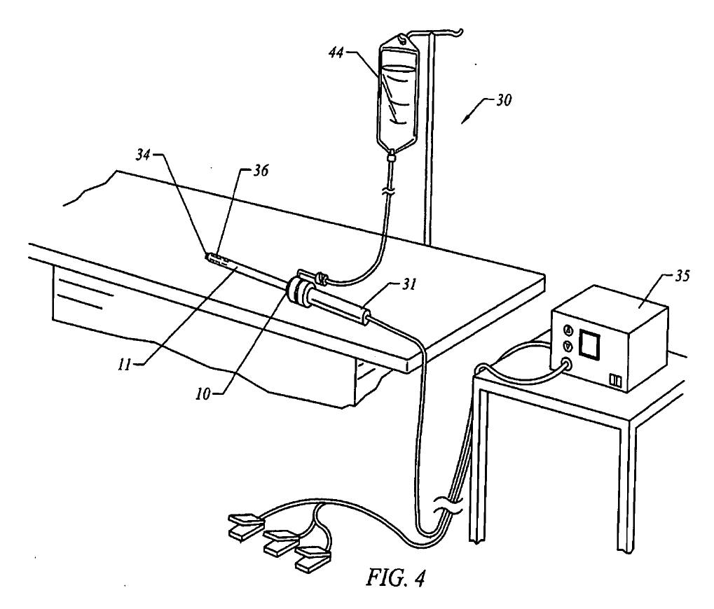

Embodiments of the present invention are illustrated in FIGS. 1, 2a, 2b, 4 and 5 wherein a pinhole is provided for controlling the supply of fluid to form a fluid bridge between the electrodes. In the embodiments illustrated in FIGS. 1 and 4, the instrument includes an elongated member (11) having a proximal end portion (15, 31) that includes electrical terminals (33) for connecting the active and return electrodes (12, 14, 34, 36) to a high frequency voltage supply (35).

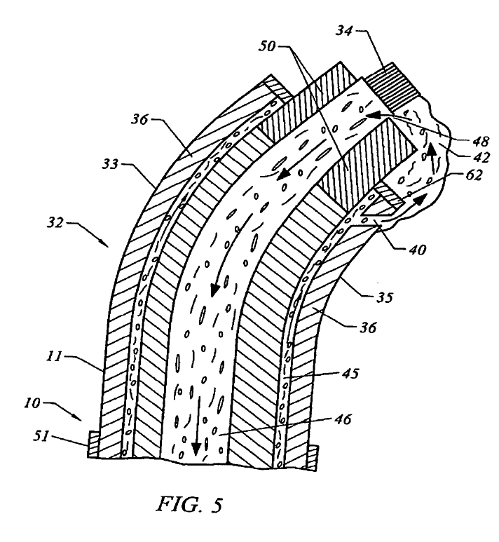

In the embodiment illustrated in FIG. 5, the instrument (10) comprises elongated member (11) having a distal end portion (32); an active electrode (34) and a return electrode (36) disposed on the distal end portion; and at least one pinhole (40) defined on the distal end portion for forming an conductive fluid bridge (42) between the active and return electrodes. Also in the embodiment of FIG. 5, the distal end portion (32) is a generally curved member having an outer curved surface (33) and an inner curved surface (35). In this embodiment, a pinhole (40) is defined by the inner curved surface. Also included in this embodiment is an electrically insulating member (50) disposed between the active and return electrodes. A vacuum system (36) is provided within a lumen in the elongated member (11) for aspirating fluid through a suction inlet (48) away from the active and return electrodes (34, 36). In alternative embodiments not shown, the pinhole is defined on the outer curved surface (33) through the insulating member (50).

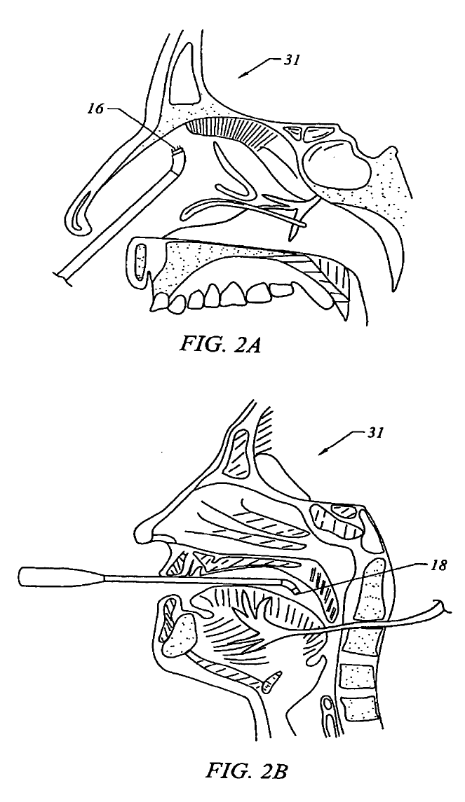

Also in the embodiment of FIG. 5, the pinhole (40) is aimed to discharge fluid towards the active electrode (34) near the return electrode (36). In this embodiment, the conductive fluid is saline or lactated ringers solution, and the fluid bridge is in the form of a bridge (42) between the active and return electrodes (32) is formed without flooding the target location, the electrodes or any other area. In this embodiment the conductive fluid bridge (42) forms a conductive path between the active (34) and return (36) electrodes regardless of the orientation of the distal end portion (32) relative to the target location as illustrated in FIGS. 2 (a) and 2(b). Although saline is shown in this embodiment, other conventional conductive fluid can be used including gels and lactated ringers solution.

The shape of the pinhole may vary widely, for example it may be circular, square, or another shape. Also the number of pinholes may vary from one to about 5-10 or more. Further, the angle or direction of the fluid through the exit of the pinhole is preferably towards the active electrode, which is generally distal relative to the return electrode. In various embodiments, the angle of the hole relative to the shaft is preferably less than 90° and is about 30-60° from the longitudinal axis of the shaft member. In various embodiments, a range of pinhole sizes is useable, however a preferred range is a diameter between about 0.015 inch to about 0.250 inch, and more preferred about 0.030 inch.

In the embodiment illustrated in FIGS. 4 and 5, the apparatus further includes a conductive fluid reservoir (44) connected to the pinhole (40) through a lumen in the elongated shaft for supplying conductive fluid to the pinhole. The lumen may be sealed at the distal end with an endcap (62) adhesive, ceramic, epoxy or another suitable means. In the configuration shown in FIGS. 4 and 5 the conductive fluid reservoir (44) height relative to the position of the distal tip of the apparatus (34) is adjustable to control the formation of the conductive fluid droplet. This height may vary preferably from about 5 cm to 3000 cm, more preferably from about 40 cm to about 100 cm.

In the embodiment of FIG. 5, the apparatus further comprises a vacuum system (46) defining a suction inlet (48) on the end portion. The vacuum system (46) in this embodiment is disposed at least partly within a lumen defined by the elongated member (11). In this embodiment a conventional vacuum system as disclosed in U.S. Pat. Nos. 5,683,366 and 6,235,020, supra, can be used.

Also as shown in FIG. 4, this embodiment of the apparatus includes a high frequency voltage supply connected to the active (34) and return (36) electrodes. Examples of various configuration of voltage supply are described in U.S. Pat. Nos. 5,683,366 and 6,235,020, incorporated herein, supra.

In a further embodiment, the invention is a method of performing a dry field surgical procedure comprising: applying ablative energy from a distal end of an elongated shaft towards a target tissue wherein the target tissue is not flooded or submerged in electrically conductive fluid; and maintaining a fluid bridge between an active electrode and a return electrode at the distal end of the shaft regardless of orientation of the electrodes. The fluid bridge is maintained by directing conductive fluid through a pinhole defined on the distal end of the shaft in accordance with the present apparatus. In various embodiments, the present method further includes generating the plasma from the electrically conductive fluid by applying a conventional high frequency voltage across the electrodes, as described, for example, in U.S. Pat. Nos. 5,683,366 and 6,235,020, supra.

In accordance with the present method, since the apparatus maintains an electrically conductive fluid bridge between the electrodes regardless of the orientation of the electrodes, the instrument can be used in dry field procedures for ablation tissues such as in the larynx, the nose, the adenoids and tonsils, as described, supra.

While the invention is described with reference to the Figures and method herein, it will be appreciated by one ordinarily skilled in the art that the invention can also be practiced with obvious modifications wherein it is desired to treat tissue using an conductive fluid bridge. Thus the scope of the invention should not be limited to the embodiments as described herein, but is limited only by the scope of the appended claims.

What is claimed is:

1. An electrosurgical apparatus for treating targeted body tissue, comprising:
an active and a return electrode;
a vacuum suction inlet located near said active electrode; and
at least one pinhole defined on said apparatus near said return electrode, wherein said pinhole and said vacuum suction inlet are adapted to maintain an electrically conductive fluid bridge between said active and return electrodes regardless of the orientation of said apparatus.

2. The apparatus of claim 1, wherein said pinhole is about 0.015 inch to about 0.250 inch in diameter.

3. The apparatus of claim 1, wherein said pinhole is about 0.030 inch in diameter.

4. The apparatus of claim 1, wherein said pinhole is aimed at said active electrode to form said conductive fluid bridge between said electrodes.

5. The apparatus of claim 1, further comprising an elongated member having a distal end portion comprised of said active and return electrodes and including an outer and an inner curved sections, wherein said pinhole is defined by said inner curved section.

6. The apparatus of claim 5, wherein said pinhole is defined by said outer curved section.

7. The apparatus of claim 1, wherein said conductive fluid bridge is selected from the group consisting of saline and lactated ringers solution.

8. The apparatus of claim 1, further comprising a fluid reservoir fluidly connected to said pinhole.

9. The apparatus of claim 8, wherein said fluid reservoir is disposed at least partly within a lumen defined by said apparatus.

10. The apparatus of claim 1, wherein said vacuum system is disposed at least partly within a lumen defined by said apparatus.

11. The apparatus of claim 1, further comprising an electrically insulating member disposed between said active electrode and said return electrode.

12. The apparatus of claim 11, wherein said return electrode defines said pinhole.

13. The apparatus of claim 12, wherein said pinhole is positioned to discharge said conductive fluid across said insulating member to either said active or return electrodes such that said conductive fluid provides a conductive bridge between said electrodes across said insulating member.

14. The apparatus of claim 1, further including a high frequency voltage supply connected to said active and return electrodes for generating plasma from said conductive fluid bridge.

15. The apparatus of claim 14, wherein said active and return electrodes are adapted to ablate body tissues in the larynx and nose.

16. An electrosurgical instrument, comprising:
an elongated shaft having a distal end portion;
an active and a return electrode disposed on said distal end portion;
a vacuum system having a suction inlet near said active electrode; and
at least one pinhole defined by said distal end portion near said return electrode such that when an electrically conductive fluid supply is coupled to said pinhole, a conductive fluid bridge is formed between said active and return electrodes regardless of the orientation of said instrument.

17. The instrument of claim 16, further including a conductive fluid system for supplying said conductive fluid to said pinhole.

18. The apparatus of claim 16, wherein said pinhole is about 0.015 inch to about 0.205 inch in diameter.

19. The apparatus of claim 16, wherein said pinhole is about 0.030 inch in diameter.

20. The apparatus of claim 16, wherein said pinhole is aimed at said active electrode to form said conductive fluid bridge with said return electrode.

21. The apparatus of claim 16, wherein said pinhole is defined in said distal end portion at an angle of less than 90° to the longitudinal axis of said distal end portion.

22. The apparatus of claim 16, wherein said pinhole is defined in said distal end portion at an angle of about 30° to 60° to the longitudinal axis of said distal end portion.

23. The apparatus of claim 16, wherein said vacuum system is disposed at least partly within a lumen defined by said distal end portion.

24. The apparatus of claim 16, wherein said conductive fluid system is disposed at least partly within a lumen defined by said distal end portion.

25. The instrument of claim 16, further comprising a high frequency voltage supply for generating plasma between said active and return electrodes.

26. The apparatus of claim 16, further comprising an electrically insulating member disposed between said active and return electrodes.

27. A method of ablating body tissue in a dry-field surgical procedure, comprising:
applying ablative energy to a target tissue not flooded or submerged with an electrically conductive material;
maintaining an electrically conductive fluid bridge between an active and a return electrode near said target tissue to generate said ablative energy regardless of the orientation of said electrodes relative to said target tissue.

28. The method of claim 27, further comprising forming said conductive fluid bridge using a pinhole to control flow of said conductive fluid near said return electrode.

29. The method of claim 28, wherein said pinhole is about 0.015 inch to about 0.250 inch in diameter.

30. The method of claim 28, wherein said pinhole is about 0.030 inch in diameter.

31. The apparatus of claim 28, further comprising aiming said pinhole at said active electrode to form said conductive fluid bridge.

32. The method of claim 28, further comprising aspirating said fluid from said conductive fluid bridge through a vacuum suction inlet positioned near said active electrode.

33. The method of claim 28, further comprising applying a high frequency voltage to said active and return electrodes to generate said plasma.

34. The method of claim 28, wherein said ablative energy comprises plasma.

35. The method of claim 28, further comprising applying said plasma to body tissues in the larynx and nose.

36. A system for ablating tissue comprising:
an apparatus including a pinhole for maintaining an electrically conductive fluid bridge between an active and a return electrode on said apparatus regardless of the orientation of said electrodes relative to said tissue;
a vacuum system for aspirating fluid from said fluid bridge;
a high-frequency voltage generator for generating plasma between said active and return electrodes; and
a conductive fluid reservoir system for maintaining a supply of said conductive fluid at said electrodes.

37. The system of claim 36, wherein said pinhole is about 0.015 inch to about 0.250 inch in diameter.

38. The system of method of claim 36, wherein said pinhole is about 0.030 inch in diameter.
